(19) United States
(12) Patent Application Publication
(10) Pub. No.: US 2023/0236184 A1
(43) Pub. Date: Jul. 27, 2023

(54) PROTEIN SENSING PLATFORM WITH A COMBINATION OF CONDUCTING POLYMERS, AROMATIC AND CONJUGATED ALDEHYDES ON A CELLULOSE PAPER BASE

(71) Applicant: Opteev Technologies, Inc., Baltimore, MD (US)

(72) Inventors: Sauvik CHATTERJEE, Dist-Hooghly (IN); Soumyo CHATTERJEE, Kolkata (IN); Biplab PAL, Ellicot City, MD (US); Conrad BESSEMER, Millersville, MD (US)

(73) Assignee: Opteev Technologies, Inc., Baltimore, MD (US)

(21) Appl. No.: 18/158,437

(22) Filed: Jan. 23, 2023

(30) Foreign Application Priority Data
Jan. 22, 2022 (IN) 202221003699

(51) Int. Cl. G01N 33/543; G01N 33/569; B01L 3/00; G01N 27/22; G01N 27/12

(52) U.S. Cl. CPC G01N 33/54373; G01N 33/56983; B01L 3/502715; G01N 27/22; G01N 27/12; B01L 2300/0645; B01L 2300/0825; G01N 2333/165

(57) ABSTRACT

Biosensors are provided for the detection of pathogens such as viruses. The sensors can includes a substrate, and a film disposed on the substrate. The film can include an electrically-conducting polymer, and an aromatic dialdehyde such as terephthaldehyde. The sensors experience a fast and repeatable decrease in electrical conductivity in the presence of certain pathogens, including the SARS-Cov-2 pseudo virus.

### BACKGROUND

The detection of proteins is a potential tool for the detection of pathogens (including viruses and bacteria), pesticides, and many other bio-fouling agents. As was exemplified during the Covid-19 pandemic, the detection of viruses and other bio-particles directly from human breath can be a fast, efficient and cost-effective way of screening potential patients and contagious carriers, or “super-spreaders.” This technique can be a quicker and less-expensive alternative to the commonly used RT-PCR based techniques.

Indian Patent Application No. 202021002188 to Mukherjee et al. (“Mukherjee”) discloses a biosensor comprising conducting polymers, such as polyaniline, polypyrrole, poly(3,4-ethylenedioxythiophene), poly(3,4-ethylenedioxythiophene)-polystyrene sulphonate, polyacetylene, poly(3,4-ethylenedioxythiophene), poly(p-phenylene vinylene) and polythiophene, along with glutaraldehyde as a linker, grown on a paper substrate. The sensing principle of the Mukherjee sensor is based on chemiresistivity. A film of conducting polymer is grown upon a paper surface by radicle polymerization, and biogenic linkers are added to create viral agglomeration in the presence of a virus. Mukherjee describes the change of impedance of the thin film sensor upon interaction with the virus.

Djaalab et al. (Catalysts, 2018, 8, 233) discloses the use of nanomaterials over conducting PANI for electrochemical biosensing of enzyme and proteins. Ramnathan et al. (Electroanalysis 1995, 7, 579) and Verghese et al. (J. Appl. Polym. Sci. 1998, 70, 1447) have reported the immobilization of GOD via manipulation of pore size in conducting polymers to improve loading parameters of enzymes.

The unstable nature of the above biosensors is a major drawback, as such instability can lead to fluctuations in the absolute chemosensitivity of the biosensors after prolonged exposure into atmosphere, making the biosensors unsuitable for production and commercial use.

A. Mulchandani et al., (Environ. Sci. Technol. 2010, 44, 9030) has reported on chemiresistive immunosensors based on single polypyrrole (Ppy) nanowire for highly sensitive, specific, label free, and direct detection of viruses. Bacteriophages T7 and MS2 were used as safe models for viruses for demonstration. Ppy nanowires were electrochemically polymerized into alumina templates. Single nanowire-based devices were assembled on a pair of gold electrodes by ac dielectrophoretic alignment, and were anchored using maskless electrodeposition. Anti-T7 or anti-MS2 antibodies were immobilized on single Ppy nanowire using EDC-NHS chemistry to fabricate a nano biosensor for the detection of corresponding bacteriophage. The biosensors showed sensitivity with a lower detection limit of 10-3 plaque forming unit (PFU) in 10 mM phosphate buffer.

### SUMMARY

The disclosed technology relates to a sensor system comprising a layered thin film of electrically-conducting polymers including, without limitation, polyaniline, functionalized polyaniline, polypyrrole, poly(3,4-ethylenedioxythiophene), poly(3,4-ethylenedioxythiophene)-polystyrene sulphonate (PEDOT:PSS), polyacetylene, poly(3,4-ethylenedioxythiophene), poly (p-phenylene vinylene), polythiophene etc., and aromatic conjugated dialdehydes, deposited on a cellulose substrate. The sensor system exhibits capacitance properties that make it suitable as a chemiresistive sensor to rapidly detect bio-proteins present in liquid and aerosolized form.

In the conventional GA-based biosensor, folding and/or twisting of alkane chains leads to crosslinking amongst the aldehyde itself, and thereby limits the sensitivity of the biosensor over time. This inherent stability issue is addressed in the disclosed technology by introducing more rigid, aromatic TA that does not undergo such crosslinking, thereby making the biosensors environmentally stable.

Owing to its completely distinct interaction mechanism, the TA-based biosensors show a higher relative change in impedance upon virus exposure, by a few orders of magnitude, than conventional GA-based sensors. It is believed that the use of TA in the polymer sensors additionally includes the capacitive contribution of the virus, resulting in such a profound change in the impedance of the sensor. This modification method of replacing TA with GA leads to a unique sensing pattern. Hence, the measurement technique disclosed herein can be envisaged as a more sensitive and effective avenue towards detection of virus.

The sensitivity of TA-based sensors shows a superior reproducibility in the detection of viruses compared to that of GA-based sensors, which can be crucial to the implementation and use of the sensors in commercial applications.

The conventional, conducting paint-based measurement technique of the disclosed technology effectively provides results from bulk studies. This techniques, therefore, is inherently slow and inadequate for instantaneous phenomenon such as virus interaction. In contrast, the probe-based surface electrical characterization route of the disclosed technology is relatively fast and efficient in tracking the instantaneous interaction between virus and sensor, and therefore holds better potential for real life virus sensing applications.

In another aspect of the disclosed technology, a sensor for detecting the presence of a pathogen includes a substrate; and a film disposed on the substrate and having an electrically-conducting polymer and an aromatic dialdehyde.

In another aspect of the disclosed technology, the aromatic dialdehyde is terephthaldehyde.

In another aspect of the disclosed technology, the electrically-conducting polymer is selected from the group including polyaniline, functionalized polyaniline, polypyrrole, poly(3,4-ethylenedioxythiophene), poly(3,4-ethylenedioxythiophene)-polystyrene sulphonate (PEDOT:PSS), polyacetylene, poly(3,4-ethylenedioxythiophene), poly (p-phenylene vinylene), and polythiophene.

In another aspect of the disclosed technology, the substrate includes cellulose.

In another aspect of the disclosed technology, the film is deposited on the substrate.

In another aspect of the disclosed technology, the probe further includes a plurality of probes disposed on a surface of the film.

In another aspect of the disclosed technology, each of the probes includes gold-coated tungsten.

In another aspect of the disclosed technology, each of the probes has a head diameter of about 50 microns.

In another aspect of the disclosed technology, a dynamic response of the sensor to the presence of a SARS-Cov-2 pseudo virus is substantially instantaneous.

In another aspect of the disclosed technology, the dynamic response of the sensor to the presence of the SARS-Cov-2 pseudo virus is a drop in conductivity.

In another aspect of the disclosed technology, a response of the sensor to the presence of the pathogen includes a capacitive contribution of the pathogen.

In another aspect of the disclosed technology, the sensor is environmentally stable.

In another aspect of the disclosed technology, the substrate is a one-dimensional substrate.

In another aspect of the disclosed technology, the aromatic dialdehyde does not undergo cross-linking.

In another aspect of the disclosed technology, the film is configured so that an impedance of the film rises when the film is exposed to the pathogen.

In another aspect of the disclosed technology, the film is developed using a radial polymerization technique.

In another aspect of the disclosed technology, a method of producing a biosensor includes soaking cellulose paper with a monomer in an acidic solution; and adding a radical initiator to form an electrically-conducting polymer film upon a surface of the paper.

In another aspect of the disclosed technology, soaking cellulose paper with a monomer in an acidic solution includes soaking the cellulose paper with aniline.

In another aspect of the disclosed technology, a method is provided for measuring the impedance of a biosensor having a substrate, and a film disposed on the substrate. The method includes placing gold coated tungsten probes having a head diameter of about 50 microns on a surface of the substrate; connecting the probes to an impedance analyzer; and applying a voltage across the probes.

In another aspect of the disclosed technology, applying a voltage across the probes includes sweeping a frequency of the voltage between about 10 Hz to about 10 kHz, with an applied bias of about 100 mV AC.

### DESCRIPTION OF THE DRAWINGS

The following drawings are illustrative of particular embodiments of the present disclosure and do not limit the scope of the present disclosure. The drawings are not to scale and are intended for use in conjunction with the explanations provided herein. Embodiments of the present disclosure will hereinafter be described in conjunction with the appended drawings.

FIG. 1 is a diagrammatical depiction of a method of synthesizing polyaniline (PANI) upon a cellulose paper substrate.

FIG. 2 depicts the chemical structures of different forms of PANI.

FIG. 3a depicts conventional (prior art) conductive paste based bulk electrical measurement technique, and a probe-based surface electrical measurement technique in accordance with the principles of the disclosed technology.

FIG. 3b are diagrammatical representations of a conventional (prior art) protein-sensing biosensor comprising glutaraldehyde, and a protein-sensing biosensor comprising terephthaldehyde in accordance with the principles of the disclosed technology.

FIG. 4a is a graphical representation of impedance vs. frequency data for GA-coated PANI sensors, at different test conditions FIG. 4b is a graphical representation of the results of a reproducibility study on the relative change in the impedance of the GA-coated PANI sensors after exposure to a virus.

FIG. 4c is a graphical representation of impedance vs. frequency data for TA-coated PANI sensors, at different test conditions

FIG. 4b is a graphical representation of the results of a reproducibility study on the relative change in the impedance of the TA-coated PANI sensors after exposure to virus.

FIG. 5 is a graphical representation of a time series response of TA-coated PANI sensor upon exposure to a virus.

FIG. 6 is a diagrammatical illustration of a conventional (prior art) two-dimensional substrate for a biosensor, and a one-dimensional substrate for a biosensor in accordance with the principles of the disclosed technology.

### DETAILED DESCRIPTION

The inventive concepts are described with reference to the attached figures, wherein like reference numerals represent like parts and assemblies throughout the several views. The figures are not drawn to scale and are provided merely to illustrate the instant inventive concepts. The figures do not limit the scope of the present disclosure or the appended claims. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

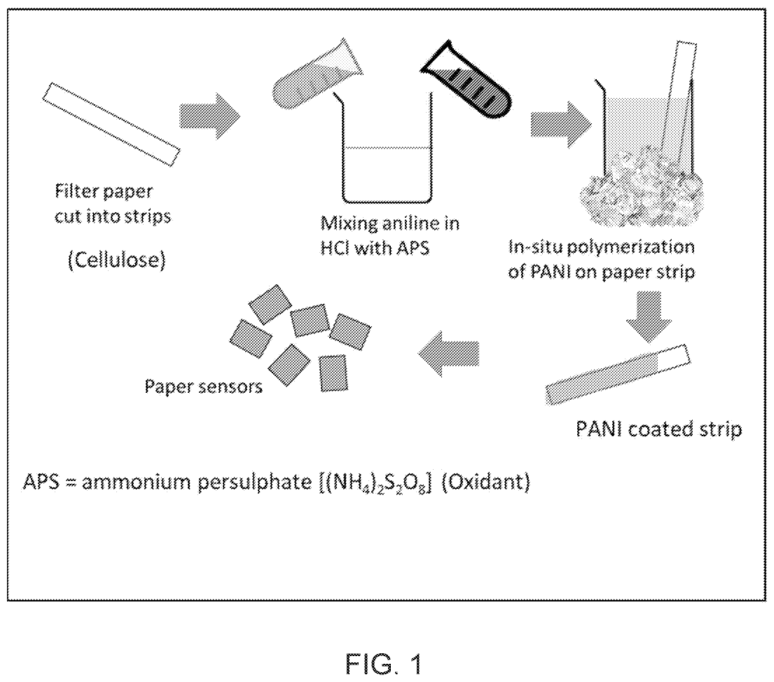

A paper-based biosensor is disclosed. The sensor is developed using a radical polymerization technique. FIG. 1 depicts a method for synthesizing polyaniline (PANI) upon a cellulose paper substrate. In an acidic solution, cellulose paper is soaked with a monomer, followed by the addition of a radical initiator, leading to the formation of an electrically-conducting polymer film upon the surface of the paper.

Polyaniline (PANI) is formed when aniline is used as the monomer. Depending upon the redox conditions, the polymers can exist in an oxidized form (pernigraniline, P-PANI), a reduced form (leucoemeraldine, L-PANI), or a partially oxidized form (emeraldine, E-PANI). Each type of form has a different electrical conductivity value, with E-PANI having the highest conductivity value in the range of 10-100 S cm⁻¹.

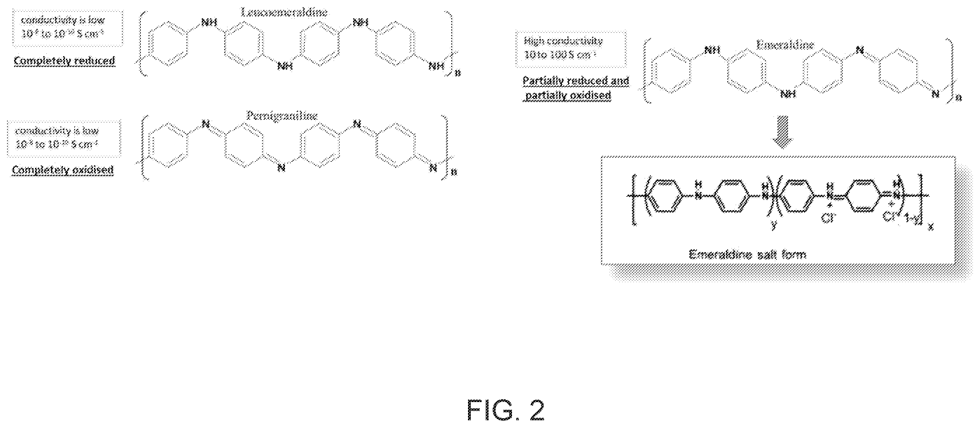

The chemical structure of different forms of PANI is depicted in FIG. 2. The conduction through the polymer depends upon the composition of the polaron and the bi-polaron. The biosensors disclosed herein have been developed with emeraldine salt, followed by coating using an aromatic aldehyde, terephthaldehyde (TA). In the prior art, the preferred aldehyde is glutaraldehyde (GA), and the sensing and/or surface functionalization were carried out immediately after the aldehyde coating. A major drawback of using GA is that it undergoes crosslinking amongst itself with time, which decreases the sensitivity of the biosensor by blocking the active sites of the biosensor.

To address the above issue, GA has been replaced with TA in the disclosed technology. Being an aromatic dialdehyde, TA is rigid and stable under atmospheric conditions for a longer time than GA. Thus, replacing GA with TA makes the sensor environmentally stable for longer period of time, which can be essential from a productization and user point of view.

### Improvement in the Experimental Technique

The prior art regarding electrical characterization of polymer biosensors, as disclosed in Mukherjee, mainly involves the use of AC impedance spectroscopy to measure the impedance of the system, where conductive paint is used to prepare the electrodes. Typically, conductive silver paint is used in such measurements and the paint, due to its liquid nature, penetrates through the cellulose network and results in the measurement of bulk properties.

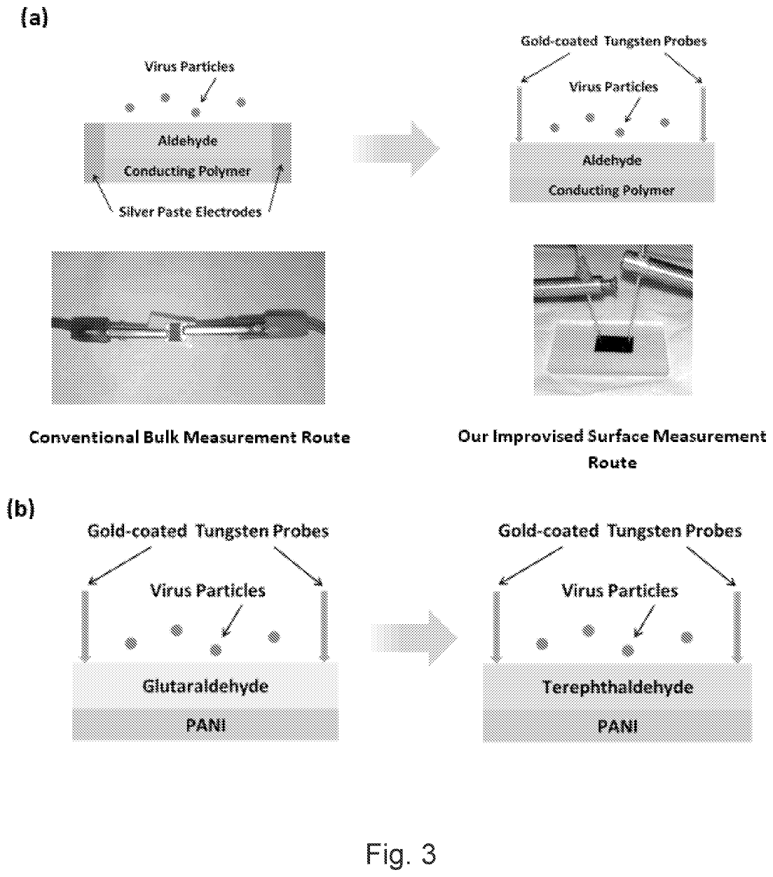

On the other hand, the incidence of virus particles on the sensor surface, and the subsequent interaction of the protein with the polymer is an instantaneous surface phenomenon followed by a slower bulk phenomenon. Hence, conventional bulk measurements not only are inadequate, but also are inappropriate to provide the actual results quickly. To overcome this limitation of conventional measurement, Applicants have opted for surface electrical measurements which are more stable, accurate, and provide results faster compared to the bulk studies (FIG. 3a).

In the modified measurement process, the impedance analyzer is connected to the biosensors by directly placing gold coated tungsten probes, having a head diameter of about 50 microns, on the surface of the polymer-coated cellulose papers. The frequency is swept from about 10 Hz to about 10 kHz with about 100 mV AC as the applied bias.

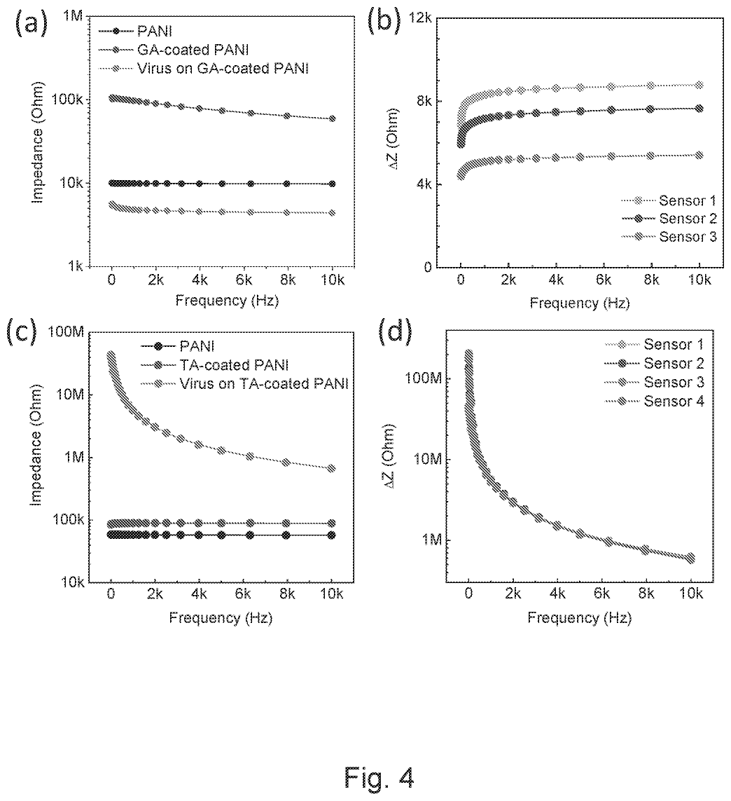

A comparison of the virus interaction behavior of glutaraldehyde (GA) coated PANI sensors and terephthaldehyde (TA) coated PANI sensors through impedance spectroscopy showed a significant change between the two systems. As shown in FIG. 4A, the GA-coated PANI sensors, when exposed to controlled virus particles, experienced a noticeable drop in impedance. On the other hand, under similar controlled exposure condition, the impedance of the TA-coated PANI sensors rose, and the relative change in the impedance was more profound in the TA-coated PANI sensors, as shown in FIG. 4c. Moreover, as can be seen in FIGS. 4b and 4d, the sensitivity of TA-based PANI sensors showed a superior reproducibility in detection of virus compared to that of GA-based sensors, which is of substantial importance to implementing the process in commercial applications.

The contradictory trend in the above-noted impedance change upon virus exposure infers a completely unique interaction mechanism between the virus and the TA-based biosensors, in relation to the GA-based biosensors. According to Shaheen et al. (BioEssays, 2021, 43, 2000312), in case of GA, the s-protein of the virus binds to the crosslinking, forming Schiff base type bonding with the spike protein and releasing the virion. In the case of TA, however, the Schiff base formation is difficult owing to the rigid and conjugated structure leading to rise in the impedance. The cellulose substrates were mainly based on 300 GSM papers having a typical width of 1 mm. As a thinner substrate inevitably reduces the capture cross-section of virus particles, the dimension was optimized to 1 mm×10 mm to achieve the required trade of between sensitivity and virus capture process.

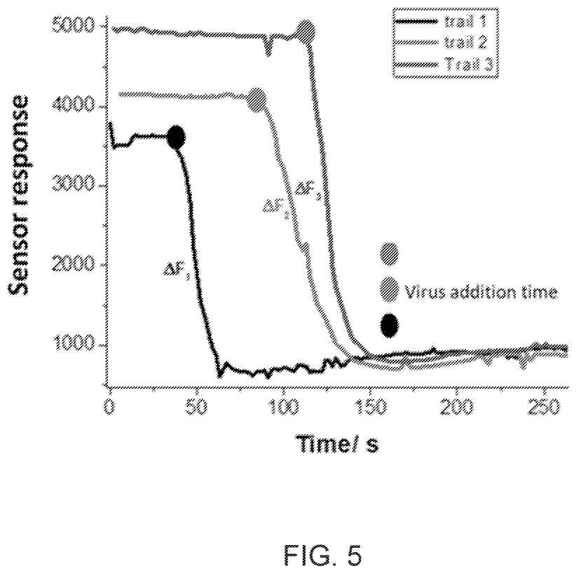

The dynamic response for the TA-coated sensors upon addition of SARS-Cov-2 pseudo virus has shown an instant drop in conductivity, as depicted in FIG. 5.

Owing to its completely distinct interaction mechanism, the TA-based biosensors show a higher relative change in impedance upon virus exposure, by a few orders of magnitude, than conventional GA-based sensors. It is believed that the use of TA in the polymer sensors additionally includes the capacitive contribution of the virus, resulting in such a profound change in the impedance of the sensor. This modification method of replacing TA with GA leads to a unique sensing pattern. Hence, the measurement technique disclosed herein can be envisaged as a more sensitive and effective avenue towards detection of virus.

The sensitivity of TA-based sensors shows a superior reproducibility in the detection of viruses compared to that of GA-based sensors, which can be crucial to the implementation and use of the sensors in commercial applications.

The conventional, conducting paint-based measurement technique of the disclosed technology effectively provides results from bulk studies. This techniques, therefore, is inherently slow and inadequate for instantaneous phenomenon such as virus interaction. In contrast, the probe-based surface electrical characterization route of the disclosed technology is relatively fast and efficient in tracking the instantaneous interaction between virus and sensor, and therefore holds better potential for real life virus sensing applications.

The two-dimensional (2D) design of conventional biosensors, although effective at detecting the presence of viruses, contains a significant number of conducting pathways. Because the sensitivity of such chemiresistive sensors depends on the change in the resistance of the sensor after interaction with the virus particles, the presence of a such large number of conducting pathways reduces the impeding effect of the virus under electrical characterization, thereby limiting the sensitivity of the sensor by lowering the relative change in resistance in response to the virus particles.

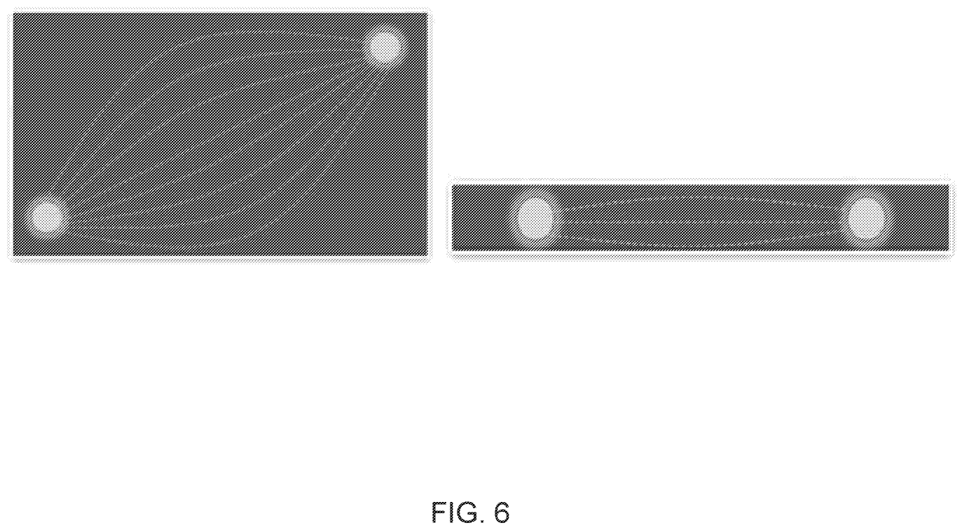

Referring to FIG. 6, to improve the sensitivity of the polymer biosensors, Applicants have developed one-dimensional (1D) cellulose-based substrates in which the width of the substrate is negligible in relation to its length. Thus, the 1D substrates inherently contain a smaller number of conducting pathways, thereby amplifying the impeding effect of the virus interaction and resulting in a better sensitivity over the conventional 2D sensors. As can be seen in FIG. 6, there are numerous conduction paths on the conventional 2D substrate (on the left side of FIG. 6), while the conduction paths are relatively limited in number on the 1D substrate (on the right side of FIG. 6).

We claim:

1. A sensor for detecting the presence of a pathogen, comprising:
a substrate; and
a film disposed on the substrate and comprising an electrically-conducting polymer and an aromatic dialdehyde.

2. The sensor of claim 1, wherein the aromatic dialdehyde is terephthaldehyde.

3. The sensor of claim 1, wherein the electrically-conducting polymer is selected from the group comprising polyaniline, functionalized polyaniline, polypyrrole, poly(3,4-ethylenedioxythiophene), poly (3,4-ethylenedioxythiophene)-polystyrene sulphonate (PEDOT:PSS), polyacetylene, poly(3,4-ethylenedioxythiophene), poly (p-phenylene vinylene), and polythiophene.

4. The sensor of claim 1, wherein the substrate comprises cellulose.

5. The sensor of claim 1, wherein the film is deposited on the substrate.

6. The sensor of claim 1, further comprising a plurality of probes disposed on a surface of the film.

7. The sensor of claim 6, wherein each of the probes comprises gold-coated tungsten.

8. The sensor of claim 6, wherein each of the probes has a head diameter of about 50 microns.

9. The sensor of claim 1, wherein a dynamic response of the sensor to the presence of a SARS-Cov-2 pseudo virus is substantially instantaneous.

10. The sensor of claim 9, wherein the dynamic response of the sensor to the presence of the SARS-Cov-2 pseudo virus is a drop in conductivity.

11. The sensor of claim 1, wherein a response of the sensor to the presence of the pathogen includes a capacitive contribution of the pathogen.

12. The sensor of claim 1, wherein the sensor is environmentally stable.

13. The sensor of claim 1, wherein the substrate is a one-dimensional substrate.

14. The sensor of claim 1, wherein the aromatic dialdehyde does not undergo cross-linking.

15. The sensor of claim 1, wherein the film is configured so that an impedance of the film rises when the film is exposed to the pathogen.

16. The sensor of claim 1, wherein the film is developed using a radial polymerization technique.

17. A method of producing a biosensor, comprising: soaking cellulose paper with a monomer in an acidic solution; and adding a radical initiator to form an electrically-conducting polymer film upon a surface of the paper.

18. The method of claim 17, wherein soaking cellulose paper with a monomer in an acidic solution comprises soaking the cellulose paper with aniline.

19. A method of measuring the impedance of a biosensor comprising a substrate, and a film disposed on the substrate, the method comprising:
placing gold coated tungsten probes having a head diameter of about 50 microns on a surface of the substrate;
connecting the probes to an impedance analyzer; and
applying a voltage across the probes.

20. The method of claim 19, wherein applying a voltage across the probes comprises sweeping a frequency of the voltage between about 10 Hz to about 10 kHz, with an applied bias of about 100 mV AC.
